(19) United States
(12) Patent Application Publication
(10) Pub. No.: US 2013/0260465 A1
(43) Pub. Date: Oct. 3, 2013

(54) CULTURE SYSTEMS

(71) Applicant: Dawn R. Applegate, San Diego, CA (US)

(72) Inventor: Dawn R. Applegate, San Diego, CA (US)

(73) Assignee: REGENEMED, INC., San Diego, CA (US)

(21) Appl. No.: 13/905,487

(22) Filed: May 30, 2013

Related U.S. Application Data
(62) Division of application No. 13/125,798, filed on Jul. 6, 2011.
(60) Provisional application No. 61/107,640, filed on Oct. 22, 2008.

(51) Int. Cl. C12N 5/00

(52) U.S. Cl. CPC C12N 5/0068
USPC 435/401

(57) ABSTRACT

A microplate is provided herein having a plate body with at least one well formed therein, the well having a first open end, a second end, an aperture being formed in the second end, and a side wall extending between the first end and the second end. The microplate further has a permeable membrane extending at least partially across the aperture formed in the second end. A microplate is provided with a permeable membrane which allows not only for removal of certain solutes from a solution (high or low molecular weight solutes), but also allows for separation of macromolecular mixtures and degradation of the membrane. The microplate is also provided with an integrated top assembly or integrated inserts. Also provided herein are methods of culturing systems in the microplates described.

### CROSS-REFERENCE

This application claims the benefit of U.S. Provisional Application No. 61/107,640, filed Oct. 22, 2008, which application is incorporated herein by reference in its entirety.

### BACKGROUND OF THE INVENTION

This invention relates to the use of containers and uses thereof for culturing systems.

### SUMMARY OF THE INVENTION

Provided herein is a device for growth of three dimensional cell or tissue cultures, comprising at least one insert configured to be received by at least one container (microplate well) such that the at least one insert is contained at least partially within the container (microplate well), each insert including a degradable, permeable bottom wall and at least one side wall connected to the bottom wall to define a discrete fluid compartment, each of said bottom walls including a porous matrix configured to permit fluid communication between each insert and a lower portion of a container (microplate well), each of said at least one insert further comprising a scaffold on top of said bottom wall.

In one embodiment, insert is capable of holding a fluid and said fluid is in communication between said each insert and a lower portion of a microplate well.

The device may comprise a multiwell insert.

In one embodiment, the scaffold comprises a three-dimensional scaffold for growth of three dimensional cell or tissue cultures.

Degradable, permeable bottom walls include, but are not limited to, cellular mesh, dextranomer microspheres, collagen, laminin, entactin, Matrigel, cotton, cellulose, granules, sheets, cloth, biodegradable microspheres, hydrogel, gauze, modified poly(saccharide)s, chitosan, starch, gelatin copolymer films, biocompatible fillers, collagen, alginate, fibrin, agarose, modified alginate, elastin, chitosan, gelatin, poly(vinyl alcohol), poly(ethylene glycol), pluronic, poly(vinylpyrollidone), hydroxyethyl cellulose, hydroxypropyl cellulose, carboxymethyl cellulose, poly(ethylene terephthalate), poly(anhydride), polypropylene fumarate), cat gut sutures, cellulose, gelatin, dextran, mixed cellulose esters, mixed cellulose esters covered by polyesters, a polymer enriched in carboxylic acid groups, multiblock copolymers of poly(ethylene oxide) (PEO) and poly(butylene terephthalate) (PBT), poly(hydroxyalkanoate)s of the PHB-PHV class, poly(esters), polylactic acid (PLA) polymers or copolymers thereof, polyglycolic acid (PGA) polymers or copolymers thereof, polylactic-co-glycolic acid (PLG) copolymers or copolymers thereof, polycaprolactone (PCL) or copolymers thereof, and/or mixtures thereof.

In one aspect, at least one insert is removable from said device. For example, a removable insert may be an unconnected insert or part of a plurality of connected inserts (an integrated top assembly).

In one embodiment, one or more inserts may be welded to the device.

In one embodiment, a bottom wall degrades after cells are attached to said scaffold and/or form a three dimensional culture. For example, a bottom wall may degrade in an amount of time from about 10 minutes to about 1 year. Alternatively, a bottom wall may degrade in an amount of time from more than about 48 hours to about 1 year.

In one embodiment, cells cultured in said insert may attach to said scaffold. Alternatively, cells cultured in said insert do not attach to said scaffold. In yet another embodiment, cells cultured in said insert integrate into said scaffold.

Provided herein is a multiwell plate apparatus, said apparatus comprising: a plurality of first containers (wells) forming a first array; a plurality of second containers (wells), forming a second array, aligned with said first array of first containers (wells); and said first and second arrays of containers (wells) coupled together and each of said plurality of second containers (wells) having a degradable, permeable bottom wall and a scaffold on top of said membrane at the interface between said first and second arrays of containers (wells).

In one embodiment, the apparatus is capable of holding a fluid and said fluid is in communication with said plurality of first and second containers (wells).

In one embodiment, a degradable, permeable bottom wall comprises a multiwell insert.

A scaffold of said apparatus may comprise a matrix for growth of three dimensional cell or tissue cultures.

Degradable, permeable bottom walls include, but are not limited to, cellular mesh, dextranomer microspheres, collagen, laminin, entactin, Matrigel, cotton, cellulose, granules, sheets, cloth, biodegradable microspheres, hydrogel, gauze, modified poly(saccharide)s, chitosan, starch, gelatin copolymer films, biocompatible fillers, collagen, alginate, fibrin, agarose, modified alginate, elastin, chitosan, gelatin, poly(vinyl alcohol), poly(ethylene glycol), pluronic, poly(vinylpyrollidone), hydroxyethyl cellulose, hydroxypropyl cellulose, carboxymethyl cellulose, poly(ethylene terephthalate), poly(anhydride), polypropylene fumarate), cat gut sutures, cellulose, gelatin, dextran, mixed cellulose esters, mixed cellulose esters covered by polyesters, a polymer enriched in carboxylic acid groups, multiblock copolymers of poly(ethylene oxide) (PEO) and poly(butylene terephthalate) (PBT), poly(hydroxyalkanoate)s of the PHB-PHV class, poly(esters), polylactic acid (PLA) polymers or copolymers thereof, polyglycolic acid (PGA) polymers or copolymers thereof, polylactic-co-glycolic acid (PLG) copolymers or copolymers thereof, polycaprolactone (PCL) or copolymers thereof, and/or mixtures thereof.

In one embodiment, an insert may be removable. Removable inserts may be a removable insert may be an unconnected insert or part of a plurality of connected inserts (an integrated top assembly).

In one embodiment, an insert may be welded to the apparatus.

In another embodiment, a bottom wall degrades after cells are attached to said scaffold and/or form a three dimensional culture. For example, a bottom wall may degrade in an amount of time from about 10 minutes to about 1 year. Alternatively, a bottom wall may degrade in an amount of time from more than about 48 hours to about 1 year.

In one embodiment, cells cultured in said second arrays of containers (wells) may attach to said scaffold. Alternatively, cells cultured in said second arrays of containers (wells) do not attach to said scaffold. In yet another embodiment, cells cultured in said second arrays of containers (wells) integrate into said scaffold.

Provided herein is the use of a permeable membrane that degrades over time and a scaffold to culture cells in a three dimensional configuration, providing: (a) a cell support means; (b) oxygen and nutrient transport means; (c) a degradable, permeable membrane; and (d) a scaffold means.

Degradable, permeable membranes may comprise part of an insert. In one embodiment, an insert may be removable. Removable inserts may be an unconnected insert or part of a plurality of connected inserts (an integrated top assembly).

In one embodiment, said second array of wells may be welded to the first arrays of wells of the apparatus.

In another embodiment, a bottom wall degrades after cells are attached to said scaffold and/or form a three dimensional culture. For example, a bottom wall may degrade in an amount of time from about 10 minutes to about 1 year. Alternatively, a bottom wall may degrade in an amount of time from more than about 48 hours to about 1 year.

In one embodiment, cells cultured in said second array of wells may attach to said scaffold. Alternatively, cells cultured in said second array of wells do not attach to said scaffold. In yet another embodiment, cells cultured in said second array of wells integrate into said scaffold.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

In a tissue culture apparatus (e.g., a insert well), a cell or tissue sample is separated from the nutrient medium by a permeable membrane. A concentration gradient of nutrients then may develop and feed the cells through this permeable membrane, which arrangement more closely reflects to the situation in vivo. The permeable membrane is attached to the bottom end of a tubular support that in turn hangs by a flange at its upper end from the top of a well containing the nutrients. The flange of the support positions the support and membrane centrally in the well. Openings in the side wall of the support provided access for a pipette to add and withdraw fluid from the well.

The present invention relates to a three-dimensional cell culture system which can be used to culture a variety of different cells and tissues in vitro for prolonged periods of time. Cells derived from a desired tissue are inoculated and grown on a pre-established stromal support matrix. The stromal support matrix comprises stromal cells, such as fibroblasts, grown to subconfluence on a three-dimensional matrix. Stromal cells may also include other cells found in loose connective tissue such as endothelial cells, macrophages/monocytes, adipocytes, pericytes, reticular cells found in bone marrow stroma, etc. The stromal matrix provides the support, growth factors, and regulatory factors necessary to sustain long-term active proliferation of cells in culture. When grown in this three-dimensional system, the proliferating cells mature and segregate properly to form components of adult tissues analogous to counterparts found in vivo.

The growth of stromal cells in three dimensions sustains active proliferation of cells in culture for longer periods of time than will monolayer systems. This may be due, in part, to the increased surface area of the three-dimensional matrix which results in a prolonged period of sub-confluency during which stromal cells actively proliferate. These proliferating sub-confluent stromal cells elaborate proteins, growth factors and regulatory factors necessary to support the long term proliferation of both stromal and tissue-specific cells inoculated onto the stromal matrix. In addition, the three-dimensionality of the matrix allows for a spatial distribution which more closely approximates conditions in vivo, thus allowing for the formation of microenvironments conducive to cellular maturation and migration. The growth of cells in the presence of this support may be further enhanced by adding proteins, glycoproteins, glycosaminoglycans, a cellular matrix, and other materials to the support itself or by coating the support with these materials.

The use of a three-dimensional support allows the cells to grow in multiple layers, thus creating the three-dimensional, cell culture system of the present invention. Bone marrow, skin, liver, pancreas and many other cell types and tissues can be grown in a three-dimensional culture system. Immortalized cell lines (e.g., Caco-2, MDCK, etc.) may also be grown in a three-dimensional culture system. The resulting cultures have a variety of applications ranging from transplantation or implantation, in vivo, of cells grown in the cultures, cytotoxicity testing and screening compounds in vitro, and the design of “bioreactors” for the production of biological materials in vitro.

The following terms used herein shall have the meanings indicated:

Adherent Layer: cells attached directly to the three-dimensional matrix or connected indirectly by attachment to cells that are themselves attached directly to the matrix.

Stromal Cells: fibroblasts with or without other cells and/or elements found in loose connective tissue, including but not limited to, endothelial cells, pericytes, macrophages, monocytes, plasma cells, mast cells, adipocytes, etc.

Tissue-Specific or Parenchymal Cells: the cells which form the essential and distinctive tissue of an organ as distinguished from its supportive framework.

Three-Dimensional Matrix: a three dimensional matrix composed of any material and/or shape that (a) allows cells to attach to it (or can be modified to allow cells to attach to it); and (b) allows cells to grow in more than one layer. This support is inoculated with stromal cells to form the three-dimensional stromal matrix.

Three-Dimensional Stromal Matrix: a three dimensional matrix which has been inoculated with stromal cells grown to sub-confluence on the matrix. The stromal matrix will support the growth of tissue-specific cells later inoculated to form the three dimensional cell culture.

Three-Dimensional Cell Culture: a three dimensional stromal matrix which has been inoculated with tissue-specific cells and cultured. In general, the tissue specific cells used to inoculate the three-dimensional stromal matrix should include the “stem” cells (or “reserve” cells) for that tissue; i.e., those cells which generate new cells that will mature into the specialized cells that form the parenchyma of the tissue.

Cells grown on a three-dimensional stromal support grow in multiple layers forming a cellular matrix. This matrix system approaches physiologic conditions found in vivo to a greater degree than monolayer tissue culture systems. The three-dimensional cell culture system is applicable to the proliferation of different types of cells and formation of a number of different tissues, including but not limited to bone marrow, skin, liver, pancreas, kidney, adrenal and neurologic tissue, to name but a few.

The culture system has a variety of applications. For example, for tissues such as skin, glands, etc. the three-dimensional culture itself may be transplanted or implanted into a living organism. Alternatively, for diffuse tissues such as bone marrow, the proliferating cells could be isolated from the culture system for transplantation. The three-dimensional cultures may also be used in vitro for cytotoxicity testing and screening compounds. In yet another application, the three-dimensional culture system may be used as a “bioreactor” to produce cellular products in quantity.

In accordance with the invention, cells derived from a desired tissue (herein referred to as tissue-specific cells or parenchymal cells) are inoculated and cultured on a pre-established three-dimensional stromal matrix. The stromal matrix comprises stromal cells grown to subconfluence on a three-dimensional matrix or network. The stromal cells comprise fibroblasts with or without additional cells and/or elements described more fully herein. The fibroblasts and other cells and/or elements that comprise the stroma may be fetal or adult in origin, and may be derived from convenient sources such as skin, liver, pancreas, etc. Such tissues and/or organs can be obtained by appropriate biopsy or upon autopsy. In fact, cadaver organs may be used to provide a generous supply of stromal cells and elements.

Fetal fibroblasts will support the growth of many different cells and tissues in the three-dimensional culture system, and, therefore, can be inoculated onto the matrix to form a “generic” stromal support matrix for culturing any of a variety of cells and tissues. However, in certain instances, it may be preferable to use a “specific” rather than “generic” stromal support matrix, in which case stromal cells and elements can be obtained from a particular tissue, organ, or individual. For example, where the three-dimensional culture is to be used for purposes of transplantation or implantation in vivo, it may be preferable to obtain the stromal cells and elements from the individual who is to receive the transplant or implant. This approach might be especially advantageous where immunological rejection of the transplant and/or graft versus host disease is likely. Moreover, fibroblasts and other stromal cells and/or elements may be derived from the same type of tissue to be cultured in the three-dimensional system. This might be advantageous when culturing tissues in which specialized stromal cells may play particular structural/functional roles; e.g., glial cells of neurological tissue, Kupffer cells of liver, etc.

Once inoculated onto the three-dimensional matrix, the stromal cells will proliferate on the matrix, achieve subconfluence, and support the growth of tissue-specific cells inoculated into the three-dimensional culture system of the invention. In fact, when inoculated with the tissue-specific cells, the three-dimensional subconfluent stromal support matrix will sustain active proliferation of the culture for long periods of time. Growth and regulatory factors may be added to the culture, but are not necessary since they are elaborated by the stromal support matrix.

The growth of the stromal cells in three dimensions will sustain active proliferation of both the stromal and tissue-specific cells in culture for much longer time periods than will monolayer systems. Moreover, the three-dimensional system supports the maturation, differentiation, and segregation of cells in culture in vitro to form components of adult tissues analogous to counterparts found in vivo.

### Three-Dimensional Cell Culture Systems

The three-dimensional stromal support, the culture system itself, and its maintenance, as well as various uses of the three-dimensional cultures are described in greater detail in U.S. Pat. Nos. 4,963,489 and 5,624,840, each of which is incorporated herein in its entirety.

A number of factors in a three-dimensional culture system are:
(a) The three-dimensional matrix provides a greater surface area for protein attachment, and consequently, for the adherence of stromal cells.
(b) Because of the three-dimensionality of the matrix, stromal cells actively grow for a much longer time than cells in monolayers before reaching confluence. The elaboration of growth and regulatory factors by replicating stromal cells during this prolonged period of subconfluency may be partially responsible for stimulating proliferation, and regulating differentiation of cells in culture.
(c) The three-dimensional matrix allows for a spatial distribution of cellular elements which is more analogous to that found in the counterpart tissue in vivo.
(d) The increase in potential volume for cell growth in the three-dimensional system may allow the establishment of localized microenvironments conducive to cellular maturation.
(e) The three-dimensional matrix maximizes cell-cell interactions by allowing greater potential for movement of migratory cells, such as macrophages, monocytes and possibly lymphocytes in the adherent layer.

### Three-Dimensional Matrices (Scaffolds)

As used herein, a three dimensional matrix (scaffold) is composed of any material and/or shape that (a) allows cells to attach to it (or can be modified to allow cells to attach to it), integrate into it, or rest on top of it (not attach); and (b) allows cells to grow in more than one layer. This support may be inoculated with stromal cells to form the three-dimensional stromal matrix.

The three-dimensional support may be of any material and/or shape that: (a) allows cells to attach to it (or can be modified to allow cells to attach to it); (b) allows cells to grow in more than one layer, and (c) is degradable over time. In one embodiment, the matrix is degradable. A variety of natural, synthetic, and biosynthetic polymers are bio- and environmentally degradable. A degradable matrix may be formed of synthetic materials, such as a synthetic material. Alternatively, a degradable matrix may include one or more biological components, particularly one or more extracellular matrix components, such as proteins and/or glycans, among others.

A multiwell or insert well insert may comprise one or more of biologic-tolerant (degradable/biodegradable) materials.

Polymers and their related copolymers and blends provide a porous structure for cell penetration and polymer degradation as scaffolds and/or extracellular matrices. For tissue regeneration, sufficient cell propagation and appropriate differentiation may be achieved in a three-dimensional cellular composite. Non-woven fabrics have been used as scaffolds in tissue applications as described in Aigner, J. et al., “Cartilage Tissue Engineering with Novel Nonwoven Structured Biomaterial Based on Hyaluronic Acid Benzyl Ester”, J. Biomed. Mater. Res., 1998, 42, 172-181; Bhat, G. S., “Nonwovens as Three-Dimensional Textiles for Composites”, Mater. Manuf. Process, 1995, 10, 67-688; Ma, T., “Tissue Engineering Human Placenta Trophoblast Cells in 3-D Fibrous Matrix: Spatial Effects on Cell Proliferation and Function”, Biotechnol. Prog., 1999, 15, 715-724, and Bhattarai, S. R. et al., “Novel Biodegradable Electrospun Membrane: Scaffold for Tissue Engineering”, Biomaterials, 2004, 25, 2595-2602; the disclosure of each of which is incorporated herein in its entirety.

Other biologic-tolerant materials include, but are not limited to, cellular mesh, dextranomer microspheres, collagen, laminin, entactin, Matrigel, cotton, cellulose, granules, sheets, cloth, biodegradable microspheres, hydrogel, gauze, modified poly(saccharide)s, chitosan, starch, gelatin copolymer films, biocompatible fillers, collagen, alginate, fibrin, agarose, modified alginate, elastin, chitosan, gelatin, poly(vinyl alcohol), poly(ethylene glycol), pluronic, poly(vinylpyrollidone), hydroxyethyl cellulose, hydroxypropyl cellulose, carboxymethyl cellulose, poly(ethylene terephthalate), poly(anhydride), polypropylene fumarate), cat gut sutures, cellulose, gelatin, dextran, mixed cellulose esters, mixed cellulose esters covered by polyesters, a polymer enriched in carboxylic acid groups, multiblock copolymers of poly(ethylene oxide) (PEO) and poly(butylene terephthalate) (PBT), poly(hydroxyalkanoate)s of the PHB-PHV class, poly(esters), polylactic acid (PLA) polymers or copolymers thereof, polyglycolic acid (PGA) polymers or copolymers thereof, polylactic-co-glycolic acid (PLG) copolymers or copolymers thereof, polycaprolactone (PCL) or copolymers thereof, and/or mixtures thereof.

A biodegradable material may be, for example, a chemical polymer or monomer that is selected from the group consisting of collagen, gelatin, polylactic acid, polyglycolic acid, polyelthylene glycol and a polyethylene glycol or a mixture thereof.

Alternatively, a biodegradable material may be, for example, a natural material selected from the group consisting of alginate, chitosan, coral, agarose, fibrin, collagen, bone, silicone, cartilage, hydroxyapatite, calcium phosphate, and mixtures thereof.

In one embodiment, a biomaterial comprises a polylactic acid (PLA) polymer, polyglycolic acid (PGA) polymer, polylactic-co-glycolic acid (PLG) copolymer biomaterial or a mixture thereof. In another embodiment, a biomaterial comprises a PLG copolymer biomaterial having a ratio of about 85 percent lactide to about 15 percent glycolide.

In one example, the biomaterial polymer is a polylactic-co-glycolic acid copolymer biomaterial and the ratio of lactide and glycolide components within the copolymer composition is altered. In another example, at least a first surface property of the polymer composition is altered. Any of the foregoing are suitable for direct use with, or for adaptation for use with, virtually any biocompatible material or device. The biocompatible materials may comprise at least a first portion that has an interconnected or open pore structure.

Especially useful aliphatic polyesters include those derived from semicrystalline polylactic acid. Polylactic acid (or polylactide) has lactic acid as its principle degradation product, which is commonly found in nature, is non-toxic and is widely used in pharmaceutical and medical industries. The polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-. By polymerizing these lactides as pure compounds or as blends, polylactide polymers may be obtained having different stereochemistries and different physical properties, including crystallinity.

Hydrogel polymers are hydrophilic, three-dimensional networks that absorb large amounts of water or biological fluids while maintaining their distinct three-dimensional structure. The coating may be a photopolarizable hydrogel polymer, or hydrated polylactic-co-glycolic acid.

Polylactic-co-glycolic acid-based hydrogel polymers have certain advantages for biological applications because of their proven biocompatibility and their demonstrated capacity to support growth and, in some cases, differentiation of cells (e.g., multipluripotent stem cells (MSCs) into multiple lineages such as bone).

Biodegradable membranes may be modified to increase or decrease the rate of degradation modifying types of polymers in said combination of polymers, ratios of polymers, using polymers having different molecular weights, or a mixture thereof.

In one non-limiting example, modifying a polymer combination comprises modifying types of polymers in said combination of polymers.

In another non-limiting example, modifying a polymer combination comprises utilizing a polymer having a different molecular weight.

In another non-limiting example, modifying a first polymer comprises utilizing a polymer in said first polymer composition to prepare said second polymer composition wherein said polymer in said second polymer composition has a different molecular weight from said polymer in said first polymer composition.

In yet another non-limiting example, modifying a first polymer comprises utilizing each polymer in said first polymer composition to prepare said second polymer composition wherein each polymer in said second polymer composition has a different molecular weight said same polymers in said first polymer composition.

A polylactide may have a high enantiomeric ratio to maximize the intrinsic crystallinity of the polymer. The degree of crystallinity of a poly(lactic acid) is based on the regularity of the polymer backbone and the ability to line crystallize with other polymer chains. If relatively small amounts one enantiomer (such as D-) is copolymerized with the opposite enantiomer (such as L-) the polymer chain becomes irregularly shaped, and becomes less crystalline. For these reasons, it may be desirable to have a poly(lactic acid) that is at least 85% of one isomer, preferably at least 90%, and most preferably at least 95% in order to maximize the crystallinity.

An approximately equimolar blend of D-polylactide and L-polylactide is also useful in the present invention. This blend forms a unique crystal structure having a higher melting point (about 210° C.) than does either the D-polylactide and L-polylactide alone (about 190° C.), and has improved thermal stability. Reference may be made to H. Tsuji et. al., Polymer, 1999, 40 6699-6708.

Copolymers, including block and random copolymers, of poly(lactic acid) with other aliphatic polyesters may also be used. Useful co-monomers include glycolide, beta-propiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxyethylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxystearic acid.

Blends of poly(lactic acid) and one or more other aliphatic polyesters, or one or more other polymers may also be used in the present invention. Examples of useful blends include poly(lactic acid) and poly(vinyl alcohol), polyethylene glycol/polysuccinate, polyethylene oxide, polycaprolactone and polyglycolide.

In blends of aliphatic polyesters with a second amorphous or semicrystalline polymer, if the second polymer is present in relatively small amounts, the second polymer will generally form a discreet phase dispersed within the continuous phase of the aliphatic polyester. As the amount of the second polymer in the blend is increased, a composition range will be reached at which the second polymer can no longer be easily identified as the dispersed, or discrete phase. Further increase in the amount of second polymer in the blend will result in two co-continuous phases, then in a phase inversion wherein the second polymer becomes the continuous phase. In one embodiment, the aliphatic polyester component forms the continuous phase while the second component forms a discontinuous, or discrete, phase dispersed within the continuous phase of the first polymer, or both polymers form co-continuous phases. Where the second polymer is present in amounts sufficient to form a co-continuous phase, subsequent orientation and microfibrillation may result in a composite article comprising microfibers of both polymers.

Useful polylactides may be prepared as described in U.S. Pat. No. 6,111,060 (Gruber, et al.); U.S. Pat. No. 5,997,568 (Liu); U.S. Pat. No. 4,744,365 (Kaplan et al.); U.S. Pat. No. 5,475,063 (Kaplan et al.); WO 98/24951 (Tsai et al.); WO 00/12606 (Tsai et al.); WO 84/04311 (Lin); U.S. Pat. No. 6,117,928 (Hiltunen et al.); U.S. Pat. No. 5,883,199 (McCarthy et al.); WO 99/50345 (Kolstad et al.); WO 99/06456 (Wang et al.); WO 94/07949 (Gruber et al.); WO 96/22330 (Randall et al.); WO 98/50611 (Ryan et al.); U.S. Pat. No. 6,143,863 (Gruber et al.); U.S. Pat. No. 6,093,792 (Gross et al.); U.S. Pat. No. 6,075,118 (Wang et al.), and U.S. Pat. No. 5,952,433 (Wang et al.), the disclosure of each U.S. patent is incorporated herein by reference in its entirety. Reference may also be made to J. W. Leenslag, et al., J. Appl. Polymer Science, 1984, 29, 2829-2842, and H. R. Kricheldorf, Chemosphere, 2001, 43, 49-54, the disclosure of each of which is incorporated herein by reference in its entirety.

In order to obtain the maximum physical properties and render the polymer film amenable to fibrillation, the polymer chains need to be oriented along two major axes (biaxial orientation). The degree of molecular orientation is generally defined by the draw ratio, that is, the ratio of the final length to the original length of the machine and transverse dimensions. The orientation may be effected by a combination of techniques including the steps of calendaring and length orienting.

Further aspects of extracellular matrix components or mixtures that may be suitable are described in U.S. patent publication numbers 20030104494 and 20030166015, each of which is incorporated by reference herein in its entirety.

Biodegradability may be adjusted by adding chemical linkages to C—C backbones of polymers; linkages such as anhydride, ester, amide bonds, etc. Degradation of PLA, PGA or PCL (or copolymers thereof) yields the corresponding hydroxy acids, making them safe for in vivo use. Other bio/environmentally degradable polymers include poly(hydroxyalkanoate)s of the PHB-PHV class, additional poly(ester)s, and natural polymers, including, but not limited to modified poly(saccharide)s (e.g., starch, cellulose, and chitosan) may be used. Chitosan is a technologically important biomaterial; chitin is the second most abundant natural polymer in the world after cellulose. Upon deacetylation, it yields chitosan, which upon further hydrolysis yields an extremely low molecular weight oligosaccharide. Chitosan possesses a wide range of useful properties including, for example, biodegradable films.

Multiblock copolymers of poly(ethylene oxide) (PEO) and poly(butylene terephthalate) (PBT) are also contemplated herein for use in three dimensional degradable matrices. Degradation rate is influenced by PEO molecular weight and content; additionally, the copolymers may be modified to adjust the rate of degradation. For example, copolymers with the highest water uptake degrade most rapidly.

In one non-limiting embodiment, a three-dimensional matrix may be made of PGA and PLA. Ratios of PGA and PLA can be adjusted to increase or decrease the rate of degradation of the matrix.

Any of these materials may be woven into a mesh, for example, to form a three-dimensional matrix.

Biodegradable matrices may be used where the three-dimensional culture is itself to be implanted in vivo.

### Inoculation of Tissue-Specific Cells onto Three-Dimensional Matrix and Maintenance of Cultures

Stromal cells comprising fibroblasts, with or without other cells and elements described below may be inoculated onto the matrix. These fibroblasts may be derived from organs, such as skin, liver, pancreas, etc. which can be obtained by biopsy (where appropriate) or upon autopsy. In fact, fibroblasts can be obtained in quantity rather conveniently from any appropriate cadaver organ. As previously explained, fetal fibroblasts can be used to form a “generic” three-dimensional stromal matrix that will support the growth of a variety of different cells and/or tissues. However, a “specific” stromal matrix may be prepared by inoculating the three-dimensional matrix with fibroblasts derived from the same type of tissue to be cultured and/or from a particular individual who is later to receive the cells and/or tissues grown in culture in accordance with the three-dimensional system of the invention.

Fibroblasts may be readily isolated by disaggregating an appropriate organ or tissue which is to serve as the source of the fibroblasts. This may be readily accomplished using techniques known to those in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase, dispase etc. Mechanical disruption can also be accomplished by a number of methods including, but not limited to the use of grinders, blenders, sieves, homogenizers, pressure cells, or insonators to name but a few. For a review of tissue disaggregation techniques, see Freshney, Culture of Animal Cells. A Manual of Basic Technique, 2 d Ed., A. R. Liss, Inc., New York, 1987, Ch. 9, pp. 107-126.

Once the tissue has been reduced to a suspension of individual cells, the suspension may be fractionated into subpopulations from which the fibroblasts and/or other stromal cells and/or elements may be obtained. This also may be accomplished using standard techniques for cell separation including but not limited to cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis and fluorescence-activated cell sorting. For a review of clonal selection and cell separation techniques, see Freshney, Culture of Animal Cells. A Manual of Basic Techniques, 2 d Ed., A. R. Liss, Inc., New York, 1987, Ch. 11 and 12, pp. 137-168.

The isolation of fibroblasts may, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. All fibroblasts will attach before other cells, therefore, appropriate stromal cells can be selectively isolated and grown. The isolated fibroblasts can then be grown to confluency, lifted from the confluent culture and inoculated onto the three-dimensional matrix (see, Naughton et al., 1987, J. Med. 18(3 & 4):219-250). Inoculation of the three-dimensional matrix with a high concentration of stromal cells, e.g., approximately 10.sup.6 to 5.times.10.sup.7 cells/ml, will result in the establishment of the three-dimensional stromal support in shorter periods of time.

In addition to fibroblasts, other cells may be added to form the three-dimensional stromal matrix required to support long term growth in culture. For example, other cells found in loose connective tissue may be inoculated onto the three-dimensional support along with fibroblasts. Such cells include but are not limited to endothelial cells, pericytes, macrophages, monocytes, plasma cells, mast cells, adipocytes, etc. These stromal cells may readily be derived from appropriate organs such as skin, liver, etc., using methods known in the art such as those discussed above. Use of liver cells in a three dimensional tissue culture system has been described in more detail in U.S. Pat. No. 5,624,840, which is incorporated herein in its entirety. In one embodiment of the invention, stromal cells which are specialized for the particular tissue to be cultured may be added to the fibroblast stroma. For example, stromal cells of hematopoietic tissue, including but not limited to fibroblasts, endothelial cells, macrophages/monocytes, adipocytes and reticular cells, could be used to form the three-dimensional subconfluent stroma for the long term culture of bone marrow in vitro. Hematopoietic stromal cells may be readily obtained from the “buffy coat” formed in bone marrow suspensions by centrifugation at low forces, e.g., 3000.times.g. Stromal cells of liver may include fibroblasts, Kupffer cells, and vascular and bile duct endothelial cells. Similarly, glial cells could be used as the stroma to support the proliferation of neurological cells and tissues; glial cells for this purpose can be obtained by trypsinization or collagenase digestion of embryonic or adult brain (Ponten and Westermark, 1980, in Federof, S. Hertz, L., eds, “Advances in Cellular Neurobiology,” Vol. 1, New York, Academic Press, pp. 209-227).

Where cultured cells are to be used for transplantation or implantation in vivo, stromal cells may be obtained from the patient's own tissues. The growth of cells in the presence of the three-dimensional stromal support matrix may be further enhanced by adding to the matrix, or coating the matrix support with proteins (e.g., collagens, elastic fibers, reticular fibers) glycoproteins, glycosaminoglycans (e.g., heparan sulfate, chondroitin-4-sulfate, chondroitin-6-sulfate, dermatan sulfate, keratan sulfate, etc.), a cellular matrix and/or other materials.

After inoculation of the stromal cells, the three-dimensional matrix may be incubated in an appropriate nutrient medium and the cells grown to subconfluence. Many commercially available media such as, but not limited to, RPMI 1640, Fisher's, Iscove's, McCoy's, and the like may be suitable for use. The three-dimensional stromal matrix may be suspended or floated in the medium during the incubation period in order to increase proliferative activity. In addition, the culture should be “fed” periodically to remove the spent media, depopulate released cells, and add fresh media.

During the incubation period, the stromal cells may grow linearly along and envelop the three-dimensional matrix before beginning to grow into the openings of the matrix. It is important to grow the cells to an appropriate degree of subconfluency prior to inoculation of the stromal matrix with the tissue-specific cells. In general, the appropriate degree of subconfluency can be recognized when the adherent fibroblasts begin to grow into the matrix openings and deposit parallel bundles of collagen.

The openings of the matrix should be of an appropriate size to allow the stromal cells to stretch across the openings and remain subconfluent for prolonged time periods. Maintaining subconfluent stromal cells which stretch across the matrix enhances the production of growth factors which are elaborated by the stromal cells, and hence will support long term cultures. For example, if the openings are too small, the stromal cells may rapidly achieve confluence, and thus, cease production of the appropriate factors necessary to support proliferation and maintain long term cultures. If the openings are too large, the stromal cells may be unable to stretch across the opening; this will also decrease stromal cell production of the appropriate factors necessary to support proliferation and maintain long term cultures. When using a mesh type of matrix, as exemplified herein, openings ranging from about 150 μm to about 220 μm may be used. However, depending upon the three-dimensional structure and intricacy of the matrix, other sizes may work equally well. In fact, any shape or structure that allow the stromal cells to stretch and maintain subconfluence for lengthy time periods will work in accordance with the invention. Cells may integrate into the matrix, attach to the matrix or not attached to the matrix.

One would recognize that cells may adhere to membranes in less than 10 minutes; therefore, degradable membranes may be used that degrade in as few as about ten minutes. In one embodiment, degradable membranes may be used that degrade in from about 10 minutes, 20 minutes, 30 minutes, 60 minutes, 3 hours, 5 hours, 10 hours, 15 hours, 24 hours, 48 hours, 72 hours, 1 week, 2 weeks, 4 weeks, 2 months, 3 months, 6 months, 8 months, 10 months or up to about 1 year, or any time frame between. Preferably, degradable membranes may be used that degrade in from about 72 hours, 1 week, 2 weeks, 4 weeks, 2 months, 3 months, 6 months, 8 months, 10 months or up to about 1 year, or any time frame between.

Biodegradable matrices (membranes) may be designed such that the rate of degradation of the membrane is adjusted based on the cell type, the end use of the cultured cells, time in culture, etc. One would understand that, for a membrane to remain substantially intact for long culture periods (e.g., one month up to about one year), the membrane may be manufactured for slow degradation upon exposure to cell and tissue culture medium. Alternatively, for a membrane to remain substantially intact for short periods of time (e.g., 10 minutes up to 1 month), the membrane may be manufactured for faster degradation upon exposure to cell and tissue culture medium. The addition or removal of one or more elements of a membrane to adjust rates of degradation is discussed in more detail above.

Membranes of the present invention may degrade at a rate such that substantially all of the cells remain in the inner well after 10 minutes. In one embodiment, a membrane degrades at a rate such that at least about 50% of the inoculated cells remain in the inner well after 10 minutes. In another embodiment, a membrane degrades at a rate such that at least about 50% of the inoculated cells remain in the inner well after 10 minutes. In another embodiment, a membrane degrades at a rate such that at least about 60% of the inoculated cells remain in the inner well after 10 minutes. In another embodiment, a membrane degrades at a rate such that at least about 70% of the inoculated cells remain in the inner well after 10 minutes. In another embodiment, a membrane degrades at a rate such that at least about 80% of the inoculated cells remain in the inner well after 10 minutes. In another embodiment, a membrane degrades at a rate such that at least about 90% or more of the inoculated cells remain in the inner well after 10 minutes.

Membranes of the present invention may degrade at a rate such that substantially all of the cells remain in the inner well after 48 hours. In one embodiment, a membrane degrades at a rate such that at least about 50% of the inoculated cells remain in the inner well after 48 hours. In another embodiment, a membrane degrades at a rate such that at least about 60% of the inoculated cells remain in the inner well after 48 hours. In another embodiment, a membrane degrades at a rate such that at least about 70% of the inoculated cells remain in the inner well after 48 hours. In another embodiment, a membrane degrades at a rate such that at least about 80% of the inoculated cells remain in the inner well after 48 hours. In another embodiment, a membrane degrades at a rate such that at least about 90% or more of the inoculated cells remain in the inner well after 48 hours.

Membranes of the present invention may degrade at a rate such that substantially all of the cells establish a three-dimensional culture on the scaffold prior to degradation of the membrane.

Membranes of the present invention may degrade at a rate such that the membrane does not begin degrading until at least about 70% of the inoculated cells begin establishing a three-dimensional culture on the scaffold. In one embodiment, a membrane degrades at a rate such that at least 70% of the inoculated cells begin establishing a three-dimensional culture on the scaffold. In another embodiment, a membrane degrades at a rate such that at least about 75% of the inoculated cells begin establishing a three-dimensional culture on the scaffold. In another embodiment, a membrane degrades at a rate such that at least about 80% of the inoculated cells begin establishing a three-dimensional culture on the scaffold. In another embodiment, a membrane degrades at a rate such that at least about 85% of the inoculated cells begin establishing a three-dimensional culture on the scaffold. In another embodiment, a membrane degrades at a rate such that at least about 90% of the inoculated cells begin establishing a three-dimensional culture on the scaffold. In another embodiment, a membrane degrades at a rate such that at least about 95% or more of the inoculated cells begin establishing a three-dimensional culture on the scaffold.

Different proportions of the various types of collagen deposited on the matrix may affect the growth of the later inoculated tissue-specific cells. For example, for optimal growth of hematopoietic cells, the matrix should preferably contain collagen types III, IV and I in an approximate ratio of 6:3:1 in the initial matrix. For three-dimensional skin culture systems, collagen types I and III are preferably deposited in the initial matrix. The proportions of collagen types deposited can be manipulated or enhanced by selecting fibroblasts which elaborate the appropriate collagen type. This can be accomplished using monoclonal antibodies of an appropriate isotype or subclass that is capable of activating complement, and which define particular collagen types. These antibodies and complement can be used to negatively select the fibroblasts which express the desired collagen type. Alternatively, the stroma used to inoculate the matrix can be a mixture of cells which synthesize the appropriate collagen types desired. The distribution and origins of the five types of collagen in more detail in Table I of U.S. Pat. No. 5,624,840.

Thus, depending upon the tissue to be cultured and the collagen types desired, the appropriate stromal cell(s) may be selected to inoculate the three-dimensional matrix.

During incubation of the three-dimensional stromal support, proliferating cells may be released from the matrix. These released cells may stick to the walls of the culture vessel where they may continue to proliferate and form a confluent monolayer. This may be prevented or minimized, for example, by removal of the released cells during feeding, or by transferring the three-dimensional stromal matrix to a new culture vessel. The presence of a confluent monolayer in the vessel may shut down the growth of cells in the three-dimensional matrix and/or culture. Removal of the confluent monolayer or transfer of the matrix to fresh media in a new vessel may restore proliferative activity of the three-dimensional culture system. Such removal or transfers should be done in any culture vessel which has a stromal monolayer exceeding 25% confluency. Alternatively, the culture system may be agitated to prevent the released cells from sticking, or instead of periodically feeding the cultures, the culture system may be set up so that fresh media continuously flows through the system. The flow rate may be adjusted to both maximize proliferation within the three-dimensional culture, and to wash out and remove cells released from the matrix, so that they will not stick to the walls of the vessel and grow to confluence. In any case, the released stromal cells can be collected and cryopreserved for future use.

Once the three-dimensional stromal matrix has reached the appropriate degree of subconfluence, the tissue-specific cells (parenchymal cells) which are desired to be cultured are inoculated onto the stromal matrix. A high concentration of cells in the inoculum may advantageously result in increased proliferation in culture much sooner than will low concentrations. The cells chosen for inoculation may depend upon the tissue to be cultured, which may include but is not limited to bone marrow, skin, liver, pancreas, kidney, neurological tissue, adrenal gland, to name but a few. In general, this inoculum should include the “stem” cell (also called the “reserve” cell) for that tissue; i.e., those cells which generate new cells that will mature into the specialized cells that form the various components of the tissue.

The parenchymal or tissue-specific cells used in the inoculum may be obtained from cell suspensions prepared by disaggregating the desired tissue using standard techniques described for obtaining stromal cells above. The entire cellular suspension itself may be used to inoculate the three-dimensional stromal support matrix. As a result, the regenerative cells contained within the homogenate will proliferate, mature, and differentiate properly on the matrix, whereas non-regenerative cells will not. Alternatively, particular cell types may be isolated from appropriate fractions of the cellular suspension using standard techniques described for fractionating stromal cells as described above. Where the “stem” cells or “reserve” cells can be readily isolated, these may be used to preferentially inoculate the three-dimensional stromal support. For example, when culturing bone marrow, the three-dimensional stroma may be inoculated with bone marrow cells, either fresh or derived from a cryopreserved sample. When culturing skin, the three-dimensional stroma may be inoculated with melanocytes and keratinocytes. When culturing liver, the three-dimensional stroma may be inoculated with hepatocytes. When culturing pancreas, the three-dimensional stroma may be inoculated with pancreatic endocrine cells. For a review of methods which may be utilized to obtain parenchymal cells from various tissues, see, Freshney, Culture of Animal Cells. A Manual of Basic Technique, 2 d Ed., A. R. Liss, Inc., New York, 1987, Ch. 20, pp. 257-288.

During incubation, the three-dimensional cell culture system may be suspended or floated in the nutrient medium. Cultures may be fed with fresh media periodically. Care should be taken to prevent cells released from the culture from sticking to the walls of the vessel where they could proliferate and form a confluent monolayer. The release of cells from the three-dimensional culture appears to occur more readily when culturing diffuse tissues as opposed to structured tissues. For example, when the three-dimensional skin culture of the invention is histologically and morphologically normal; the distinct dermal and epidermal layers do not release cells into the surrounding media. By contrast, the three-dimensional bone marrow cultures of the invention release mature non-adherent cells into the medium much the way such cells are released in marrow in vivo.

Growth factors and regulatory factors need not be added to the media since these types of factors are elaborated by the three-dimensional subconfluent stromal cells. However, the addition of such factors, or the inoculation of other specialized cells may be used to enhance, alter or modulate proliferation and cell maturation in the cultures. The growth and activity of cells in culture can be affected by a variety of growth factors such as, for example, insulin, growth hormone, somatomedins, colony stimulating factors, erythropoietin, epidermal growth factor, hepatic erythropoietic factor (hepatopoietin) and liver-cell growth factor. Other factors which regulate proliferation and/or differentiation include, for example, prostaglandins, interleukins, and naturally-occurring chalones.

### Devices

The present invention in one aspect is a insert well device having a degradable, porous membrane. Insert well devices are also known as multiwell insert plates; the two devices are referred to herein interchangeably. The portion of the device used to support the growth of cells which typically is a membrane, is detachably secured to the portion of the device used to suspend the membrane within a well containing growth medium. This arrangement affords easy manipulation of the cultured cells.

A two-piece insert well has two components, a cell retention element and a hanger for suspending the cell retention element at a preselected location within a well. The retention element is detachably secured to the bottom portion of the hanger. The cell retention element includes a porous membrane growth surface. The hanger is constructed and arranged such that it may be suspended from the periphery of the well, with a bottom portion of the hanger extending into the well. When the hanger is suspended from the periphery of the well, the retention element is suspended horizontally within the well at a preselected location within the well.

In one embodiment, the retention element is secured to the bottom of the hanger by a friction fit. In another embodiment, the retention element is hung from the hanger.

The hanger preferably includes an outwardly extending flange which is stepped so that it may hang upon the upper end of a well in a tissue culture cluster dish. The stepped flange prevents the hanger from shifting laterally within the well, thereby keeping the side-walls of the hanger spaced from the side walls of the well so as to prevent capillary action of fluid between the side walls. Capillary action is further prevented in one embodiment by the use of a funnel-shaped hanger which further removes the side walls of the hanger from the side-walls of the well. The flange is discontinuous to provide an opening which allows a pipette to be inserted into the space between the hanger and the side-walls of the well to provide access to the medium within the well.

Another aspect of the invention is the retention element itself. The retention element preferably has a side wall defining an interior and a peripheral lip extending from the side-wall. A membrane is attached to the bottom surface of the side-wall forming a tissue or cell growth support. The peripheral lip permits easy manipulation of the retention element, as well as providing structure which permits the use of the retention element in certain flow devices, described in greater detail below.

According to another aspect of the invention, both the retention element and the bottom of the hanger carry porous membranes. In this instance, an isolated growth chamber is provided between a pair of membranes.

Still another aspect of the invention is a cluster dish having a plurality of wells containing the tissue culture device as described above.

It is an object of this invention to provide a tissue or cell culture device capable of being placed in a cluster dish such that nutrients are provided to tissues or cells while allowing access to the wells in the cluster dish for the addition or removal of media.

Another object of this invention is to provide a device capable of allowing nutrients to pass to the tissues or cells in a manner which approximates the way in which nutrients pass to the cells or tissues within the human body, e.g. which allows the surfaces of the cells attached to a growth surface to receive nutrients via that growth surface.

Another object of this invention is to provide a insert well with a growth surface that is easily detachable from the insert well.

It is yet another object of this invention to provide a cell or tissue culture device having a retention element which can be removed and placed in a flow device.

In one aspect, provided herein is a device having one membrane for forming a first chamber for growing cells or tissues separate from a second chamber. In another aspect, provided herein is a device having two or more membranes for forming a first chamber for growing cells or tissues separate from a second chamber. In one embodiment, the two or more membranes are spaced apart. In another embodiment, the two or more membranes are not spaced art. One or more membranes, when used in this manner, may be modified; for example, membranes may be modified such that a vacuum may be used to suction fluid from said chamber without disturbing cells or tissues in the chamber.

Also provided herein is, for example, a device capable of spacing the side walls of a retention element a sufficient distance apart from the side-walls of a well in a cluster dish thereby preventing wicking of fluid between the respective side walls via capillary action.

Membrane scaffolds may be cut using conventional means including, but mot limited to, a punch and hammer, a laser, or any other means for cutting membranes. The diameters of the membrane may be slightly larger than, equal to, or slightly smaller than, the diameter of the inside of the insert wells. Membranes may be press fit into the wells with a convex shape. Alternatively, an O-ring may be made to fit the inside of the insert well which holds the membranes (scaffolds) to the bottom of the insert well. The O-ring, may be square cut and tall, and press against the side walls of the insert well and down on the membrane, thereby preventing the scaffold from moving. An O-ring may be selected such that it takes up as little circumference/circular surface area of the insert well inner area as possible.

Insert wells may have a thin membrane welded to the bottom of the insert well. In one embodiment, two pieces of three-dimensional scaffold may be spot-welded together. One or more welds may be used to spot wells pieces of scaffold together and the number of welds may be determined by one in the art. In one embodiment, one or more scaffolds are welded to the bottom of the insert well (insert). In another embodiment, a membrane may be molded to the bottom of the scaffold. A second weld may be peelable from the scaffold as at the end of cell/tissue growth, it may be desirable to separate it for conducting testing or for implanting in vivo.

In one aspect, a membrane used in the device has a pore size such that cells may not move across the membrane. In another aspect, a membrane used in the device has a pore size such that cells may move across the membrane. Movement, or non-movement, of cells may be determined by pore size of the membrane. In one embodiment, pore size may be from about 10 μm to about 20 μm, or any amount there between. In one embodiment, pore size may be from about 12 μm to about 18 μm, or any amount there between.

Membranes may vary in porosity. Porosity includes, for example from about 0.1 μm to about 100 μm, or any amount there between.

In one embodiment, a insert well plate is designed for removal of one insert well (insert) at a time. Alternatively, a insert well plate is designed with an integrated top assembly, thereby allowing removal of all wells at the same time, such as with a robot (called high throughput screening (HTS)).

Plates can be of any configuration of wells including, but not limited to, 1-well plates, 6-well plates, 12-well plates, 24-well plates, 32-well plates, 48-well plates or 96 well plates.

Other devices and portions thereof that may be used in the present invention include those described in, for example, U.S. Pat. Nos. 6,972,184; 5,037,656; 7,338,773; 7,429,492; 5,763,255; 5,741,701; 5,139,951; 5,272,083; 7,128,878; 4,828,386; and 5,731,417, US Application Publication Nos. 2004/0091397; 2007/0280860; 2007/0269850; and PCT Publication Nos. WO04044120; WO920927063; WO08005244; 2008/0003670; WO04044120; each of which is incorporated by reference herein in its entirety. Other devices that are not specifically disclosed herein but which may be used for three-dimensional cell culture are also contemplated herein.

It is yet another object of this invention to provide a cell or tissue device capable of growing cells in suspension, attached to said membranes or integrated within said membranes.

Provided herein is a device for growth of three dimensional cell or tissue cultures, comprising at least one insert configured to be received by at least one container (microplate well) such that the at least one insert is contained at least partially within the container (microplate well), each insert including a degradable, permeable bottom wall and at least one side wall connected to the bottom wall to define a discrete fluid compartment, each of said bottom walls including a porous matrix configured to permit fluid communication between each insert and a lower portion of a container (microplate well), each of said at least one insert further comprising a scaffold on top of said bottom wall.

In one embodiment, insert is capable of holding a fluid and said fluid is in communication between said each insert and a lower portion of a container (microplate well).

The device may comprise a multiwell insert.

In one embodiment, the scaffold comprises a three-dimensional scaffold for growth of three dimensional cell or tissue cultures.

Degradable, permeable bottom walls include, but are not limited to, cellular mesh, dextranomer microspheres, collagen, laminin, entactin, Matrigel, cotton, cellulose, granules, sheets, cloth, biodegradable microspheres, hydrogel, gauze, modified poly(saccharide)s, chitosan, starch, gelatin copolymer films, biocompatible fillers, collagen, alginate, fibrin, agarose, modified alginate, elastin, chitosan, gelatin, poly(vinyl alcohol), poly(ethylene glycol), pluronic, poly(vinylpyrollidone), hydroxyethyl cellulose, hydroxypropyl cellulose, carboxymethyl cellulose, poly(ethylene terephthalate), poly(anhydride), polypropylene fumarate), cat gut sutures, cellulose, gelatin, dextran, mixed cellulose esters, mixed cellulose esters covered by polyesters, a polymer enriched in carboxylic acid groups, multiblock copolymers of poly(ethylene oxide) (PEO) and poly(butylene terephthalate) (PBT), poly(hydroxyalkanoate)s of the PHB-PHV class, poly(esters), polylactic acid (PLA) polymers or copolymers thereof, polyglycolic acid (PGA) polymers or copolymers thereof, polylactic-co-glycolic acid (PLG) copolymers or copolymers thereof, polycaprolactone (PCL) or copolymers thereof, and/or mixtures thereof.

In one aspect, at least one insert is removable from said device. For example, a removable insert may be an unconnected insert or part of a plurality of connected inserts (an integrated top assembly).

In one embodiment, one or more inserts may be welded to the device.

In one embodiment, a bottom wall degrades after cells are attached to said scaffold and/or form a three dimensional culture. For example, a bottom wall may degrade in an amount of time from about 10 minutes to about 1 year. Alternatively, a bottom wall may degrade in an amount of time from more than about 48 hours to about 1 year.

In one embodiment, cells cultured in said insert may attach to said scaffold. Alternatively, cells cultured in said insert do not attach to said scaffold. In yet another embodiment, cells cultured in said insert integrate into said scaffold.

Provided herein is a multiwell plate apparatus, said apparatus comprising: a plurality of first wells forming a first array; a plurality of second containers (wells), forming a second array, aligned with said first array of first wells; and said first and second arrays of containers (wells) coupled together and each of said plurality of second containers (wells) having a degradable, permeable bottom wall and a scaffold on top of said membrane at the interface between said first and second arrays of containers (wells).

In one embodiment, the apparatus is capable of holding a fluid and said fluid is in communication with said plurality of first and second containers (wells).

In one embodiment, a degradable, permeable bottom wall comprises a multiwell insert.

A scaffold of said apparatus may comprise a matrix for growth of three dimensional cell or tissue cultures.

Degradable, permeable bottom walls include, but are not limited to, cellular mesh, dextranomer microspheres, collagen, laminin, entactin, Matrigel, cotton, cellulose, granules, sheets, cloth, biodegradable microspheres, hydrogel, gauze, modified poly(saccharide)s, chitosan, starch, gelatin copolymer films, biocompatible fillers, collagen, alginate, fibrin, agarose, modified alginate, elastin, chitosan, gelatin, poly(vinyl alcohol), poly(ethylene glycol), pluronic, poly(vinylpyrollidone), hydroxyethyl cellulose, hydroxypropyl cellulose, carboxymethyl cellulose, poly(ethylene terephthalate), poly(anhydride), polypropylene fumarate), cat gut sutures, cellulose, gelatin, dextran, mixed cellulose esters, mixed cellulose esters covered by polyesters, a polymer enriched in carboxylic acid groups, multiblock copolymers of poly(ethylene oxide) (PEO) and poly(butylene terephthalate) (PBT), poly(hydroxyalkanoate)s of the PHB-PHV class, poly(esters), polylactic acid (PLA) polymers or copolymers thereof, polyglycolic acid (PGA) polymers or copolymers thereof, polylactic-co-glycolic acid (PLG) copolymers or copolymers thereof, polycaprolactone (PCL) or copolymers thereof, and/or mixtures thereof.

In one embodiment, an insert may be removable. Removable inserts may be a removable insert may be an unconnected insert or part of a plurality of connected inserts (an integrated top assembly).

In one embodiment, an insert may be welded to the apparatus.

In another embodiment, a bottom wall degrades after cells are attached to said scaffold and/or form a three dimensional culture. For example, a bottom wall may degrade in an amount of time from about 10 minutes to about 1 year. Alternatively, a bottom wall may degrade in an amount of time from more than about 48 hours to about 1 year.

In one embodiment, cells cultured in said second arrays of containers (wells) may attach to said scaffold. Alternatively, cells cultured in said second arrays of containers (wells) do not attach to said scaffold. In yet another embodiment, cells cultured in said second arrays of containers (wells) integrate into said scaffold.

### Uses of the Three-Dimensional Culture System

The three-dimensional liver culture system of the invention can be used in a variety of applications. These include but are not limited to transplantation or implantation of the cultured cells in vivo; screening cytotoxic compounds, carcinogens, mutagens growth/regulatory factors, pharmaceutical compounds, etc., in vitro; elucidating the mechanism of certain diseases; studying the mechanism by which drugs and/or growth factors operate; diagnosing and monitoring cancer in a patient; gene therapy; and the production of biologically active products, to name but a few.

For transplantation or implantation in vivo, either the PC obtained from the culture or the entire three-dimensional culture could be implanted, depending upon the need. Three-dimensional-tissue culture implants may, according to the invention, be used to replace or augment existing tissue, to introduce new or altered tissue, or to join together biological tissues or structures. For example, three-dimensional liver tissue implants may be used to correct metabolic deficiencies due to single gene defects in neonates such as ornithine transcarbamylase deficiency, or to augment liver function in cirrhosis patients.

The three-dimensional cultures (e.g., liver cultures) may be used in vitro to screen a wide variety of compounds, such as cytotoxic compounds, growth/regulatory factors, pharmaceutical agents, etc. To this end, the cultures are maintained in vitro and exposed to the compound to be tested. The activity of a cytotoxic compound can be measured by its ability to damage or kill cells in culture. This may readily be assessed by vital staining techniques. The effect of growth/regulatory factors may be assessed by analyzing the cellular content of the matrix, e.g., by total cell counts, and differential cell counts. This may be accomplished using standard cytological and/or histological techniques including the use of immunocytochemical techniques employing antibodies that define type-specific cellular antigens. The effect of various drugs on normal cells cultured in the three-dimensional system may be assessed. For example, drugs that affect cholesterol metabolism, by lowering cholesterol production, could be tested on the three-dimensional liver system.

The three-dimensional cell cultures may also be used to aid in the diagnosis and treatment of malignancies and diseases. In one non-limiting example, a biopsy of liver tissue may be taken from a patient suspected of having a malignancy. If the biopsy cells are cultured in the three-dimensional system of the invention, malignant cells may be clonally expanded during proliferation of the culture. This will increase the chances of detecting a malignancy and, therefore, increase the accuracy of the diagnosis. Hepatitis virus-infected liver cells may be grown in the culture system of the invention. Moreover, the patient's culture could be used in vitro to screen cytotoxic and/or pharmaceutical compounds in order to identify those that are most efficacious; i.e. those that kill the malignant or diseased cells, yet spare the normal cells. These agents could then be used to therapeutically treat the patient.

The three-dimensional culture system of the invention may afford a vehicle for introducing genes and gene products in vivo for use in gene therapies. For example, using recombinant DNA techniques, a gene for which a patient is deficient could be placed under the control of a viral or tissue-specific promoter. The recombinant DNA construct containing the gene could be used to transform or transfect a host cell which is cloned and then clonally expanded in the three-dimensional culture system. The three-dimensional culture which expresses the active gene product, could be implanted into an individual who is deficient for that product.

In a further embodiment of the invention, three-dimensional cultures may be used to facilitate gene transduction. For example, and not by way of limitation, three-dimensional cultures of stroma comprising a recombinant virus expression vector may be used to transfer the recombinant virus into cells brought into contact with the stromal tissue, thereby simulating viral transmission in vivo. The three-dimensional culture system is a more efficient way of accomplishing gene transduction than are current techniques for DNA transfection.

In yet another embodiment of the invention, the three-dimensional culture system could be used in vitro to produce biological products in high yield. For example, a cell which naturally produces large quantities of a particular biological product (e.g., a growth factor, regulatory factor, peptide hormone, antibody, etc.), or a host cell genetically engineered to produce a foreign gene product, could be clonally expanded using the three-dimensional culture system in vitro. If the transformed cell excretes the gene product into the nutrient medium, the product may be readily isolated from the spent or conditioned medium using standard separation techniques (e.g., HPLC, column chromatography, electrophoretic techniques, to name but a few). A “bioreactor” could be devised which would take advantage of the continuous flow method for feeding the three-dimensional cultures in vitro. Essentially, as fresh media is passed through the three-dimensional culture, the gene product will be washed out of the culture along with the cells released from the culture. The gene product could be isolated (e.g., by HPLC column chromatography, electrophoresis, etc.) from the outflow of spent or conditioned media.

Uses of the three-dimensional culture system of the invention, including, but not limited to, methods of obtaining cells, methods of establishing a three-dimensional stromal matrix, methods of enhancing the growth of cells, methods of long term growth of three-dimensional cultures, methods of monitoring patients, screening compounds, three-dimensional skin culture, establishment of the three-dimensional stromal support and formation of the dermal equivalent, inoculation of a dermal equivalent with epidermal cells, morphological characterization three-dimensional skin culture, and transplantation or engraftment, and establishment of long term bone marrow cultures for human, non-human primate (macaque), and rat. Also provided herein are in vitro uses of the three-dimensional skin culture. Such in vivo and in vitro methods are described in more detail in U.S. Pat. Nos. 4,963,489 and 5,624,840, each of which is incorporated herein it its entirety.

Provided herein is the use of a permeable membrane that degrades over time and a scaffold to culture cells in a three dimensional configuration, providing: (a) a cell support means; (b) oxygen and nutrient transport means; (c) a degradable, permeable membrane; and (d) a scaffold means.

Degradable, permeable membranes may comprise part of an insert. In one embodiment, an insert may be removable. Removable inserts may be an integrated top assembly or a single insert well.

In one embodiment, said second array of wells may be welded to the first arrays of wells of the apparatus.

In another embodiment, a bottom wall degrades after cells are attached to said scaffold and/or form a three dimensional culture. For example, a bottom wall may degrade in an amount of time from about 10 minutes to about 1 year. Alternatively, a bottom wall may degrade in an amount of time from more than about 48 hours to about 1 year.

In one embodiment, cells cultured in said second array of wells may attach to said scaffold. Alternatively, cells cultured in said second array of wells do not attach to said scaffold. In yet another embodiment, cells cultured in said second array of wells integrate into said scaffold.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-29. (canceled)

30. Use of a permeable membrane that degrades over time and a scaffold to culture cells in a three dimensional configuration, providing:
(a) a cell support means;
(b) oxygen and nutrient transport means; and
(c) a degradable, permeable membrane.

31. The use of claim 30, wherein said degradable, permeable membrane comprises a bottom wall of an insert.

32. The use of claim 31, wherein said insert is removable from a device or apparatus.

33. The use of claim 32, wherein said removable insert is an unconnected insert or part of a plurality of connected inserts.

34. The use of claim 31, wherein said insert is welded to a device or an apparatus.

35. The use of claim 30, wherein said cell support means comprises a scaffold on top of said degradable, permeable membrane.

36. The use of claim 35, wherein said membrane degrades after cells are attached to said scaffold and/or form a three dimensional culture.

37. The use of claim 30, wherein said membrane degrades in an amount of time from about 10 minutes to about 1 year.

38. The use of claim 30, wherein said membrane degrades in an amount of time from more than about 48 hours to about 1 year.

39. The use of claim 35, wherein cells cultured on said cell support means attach to said scaffold.

40. The use of claim 35, wherein cells cultured on said cell support means integrate into said scaffold.

41. The use of claim 35, wherein cells cultured on said cell support means do not attach to said scaffold.
